(19) United States
(12) Patent Application Publication
(10) Pub. No.: US 2005/0033451 A1
(43) Pub. Date: Feb. 10, 2005

(54) Prosthetic foot

(76) Inventors: Michael Aigner, (US); James Auxier, (US); Jim Maltese, (US); Jnyan Patel, (US)

Correspondence Address: BOZICEVIC, FIELD & FRANCIS LLP, 1900 UNIVERSITY AVE, SUITE 200, EAST PALO ALTO, CA, 94303, US

(21) Appl. No.: 10/637,810

(22) Filed: Aug. 7, 2003

(51) Int. Cl. A61F 2/66

(52) U.S. Cl. 623/53; 623/55

(57) ABSTRACT

Features for a high performance prosthetic foot are provided in a package suitable for use with Symes and reverse-Symes amputees. The foot has a very low profile despite incorporating ankle-type torsional features complete with biasing. Other optional features are presented by way of a dual-rate padding spring system, that convincingly allow for both walking and running (together with related tasks like jumping) by way of a single prosthetic setup. Additional features may be provided in the padding springs directed toward more natural foot movement and response. The foot may be provided alone, in connection with a prosthetic socket and be bare or encased in order to appear more natural.

### FIELD OF THE INVENTION

This invention involves features for a prosthetic foot. Such features are provided to enable more effective use and improved prosthetic capability to handle diverse situations, including athletic applications.

### BACKGROUND OF THE INVENTION

In recent years, prosthetic component design has been the subject of considerable improvement. Modern materials and progressive views regarding activity as may be achieved by those in need of prosthetic devices have fueled the innovation.

As of yet, however, no prosthetic foot design has offered the sort of adaptability in feature options as taught by the present invention. The invention preferably offers advantages as described below as well as others as may be apparent to those with skill in the art, all in a design applicable to Symes and Reverse-Symes type amputations. Even in pediatric applications where components must be proportionately smaller that as applicable to adults of average size or better.

A typical Symes amputation involves an ankle disarticulation with removal of the malleoli and forward rotation of the heel pad of the end of the residual tibia. Essentially, everything below and including the talus is removed, and the Achilles tendon is removed from the calcaneus very close to its insertion point. The fat pad in the heel is then used to cover the end of the stump so that, after healing, the amputee will be able to use the appendage for some load bearing. A reverse Symes amputation removes the foot similarly. The procedure differs in that the fat pad from the heel is not used to cover the stump; instead; skin from the top of the foot is folded over it. This skin does not have an underlying fad pad and is more sensitive than the skin from the bottom of the foot, as might be expected. As a result, little or no load bearing capacity is provided by the appendage. A reverse-Symes operation is, however, appropriate in situations involving problems with the heel, such as cancer.

To accommodate what little space is available to fit a prosthetic to a Symes or Reverse Symes amputee (hereinafter, the procedures generically being referred to as Symes-type amputations) a low-profile device is required. Yet, the solution offered by the present invention is not merely low-profile. Its construction, configuration and various options features offers superb functionality as will be apparent upon review of the text below and as may be further appreciated by those with skill in the art.

### SUMMARY OF THE INVENTION

The present invention includes features offering improvement over known foot prosthetics, particularly those used to handle foot amputations (especially, any Symes-type amputation). Certain features provide an ability to effectively handle walking and running applications without changing-out components or otherwise modifying the subject device. A dual-spring system handles challenges arising from the dissimilar forcing requirements presented by each activity. Preferably, this is accomplished using a pair of elongated carbon-fiber leaf springs affixed to a centrally-located common mount, with ends of the spring elements diverging from one another. The ground-side spring, or both springs together act as the sole of the foot.

This configuration provides additional spring return force from the upper spring to assist the action of the lower spring upon deep spring compression when higher forces are applied to the foot by running or jumping. Otherwise, the lower/ground-side spring acts mostly alone. This spring system and other optional features, including adaptation enabling prosthetic foot torsion and/or inversion-eversion, each can be utilized in producing a prosthesis that closely reproduces the performance characteristics of an organic foot.

Torsional capability for the foot is provided by a rotor-stator combination with force return enabled by elastic members positioned between opposing vanes. The rotor section is mounted or adapted to be mounted to a socket for receiving the user's limb. The stator section is formed in the base of the design that is associated with sole features (i.e., toe/heel, etc.) of the foot.

Inversion-eversion capabilities are preferably provided in the prosthetic foot by a split toe and heel design in at least the ground-side spring member. Alone, or together with the split-toe features, an hour-glass shape may be used in the ground-side spring to facilitate inversion-eversion spring forcing and return.

The present invention most preferably includes all of the above-referenced features. Such an aim is, in essence, possible because of the configuration of the prosthetic foot base. It includes the torsional-design features as well a base configuration adapted to mount and provide clearance for the desired leaf-spring members.

Through use of these various tools offered by the present invention, prosthetic foot performance can be specifically tailored to meet a subject's needs. That is, it is possible to tune the shape, size or spring rate of the various active elements noted above in order to best fit a prospective user's needs. Of course, such tuning methodology as well as the noted hardware form aspects of the invention

Modularity of the various elements described in the system design facilitates iterative tuning such as by altering torsion zone stiffness/spring rate and/or the spring rate, overlap, shape or number of spring used for the sole of the prosthetic foot. The modularity also offers benefits regarding servicing and upgrade—in the latter case, especially to account for changing needs of a patient. Of course, the prosthetic foot may be used as shown in the various figures or alternatively encased in polymer or another material to provide a more natural appearance.

### BRIEF DESCRIPTION OF THE DRAWINGS

Each of the following figures diagrammatically illustrate aspects of the present invention. Like elements in the various figures are represented by identical numbering. Some such numbering is omitted, however, for the sake of clarity. Variation of the invention from that shown in the figures is contemplated.

FIG. 1 is side view of components of the inventive system, including a socket and a prosthetic foot.

FIGS. 2A and 2B are top and bottom views, respectively, of the foot assembly of the invention.

FIGS. 3A and 3B are front and rear views, respectively, of the foot assembly of the invention. is a perspective view of the components in FIG. 1 as assembled.

FIG. 4A is an exploded perspective view of the base of the inventive foot; FIGS. 4B and 4C are side and top views, respectively, of the same.

### DETAILED DESCRIPTION OF THE INVENTION

Before the present invention is described in such detail, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims made herein. Furthermore, where a range of values is provided, it is understood that every intervening value, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention. Also, it is contemplated that any optional feature of the inventive variations described herein may be set forth and claimed independently, or in combination with any one or more of the features described herein.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All existing subject matter mentioned herein (e.g., publications, patents, patent applications and hardware) is incorporated by reference herein in its entirety. The referenced items are provided solely for their disclosure prior to the filing date of the present application. Nothing herein is to be construed as an admission that the present invention is not entitled to antedate such material by virtue of prior invention.

It is noted that as used herein and in the appended claims, the singular forms “a”, “and”, “said” and “the” include plural referents unless the context clearly dictates otherwise. Conversely, it is contemplated that the claims may be so-drafted to exclude any optional element. This statement is intended to serve as antecedent basis for use of such exclusive terminology as “solely,” “only” and the like in connection with the recitation of claim elements or by use of a “negative” limitation

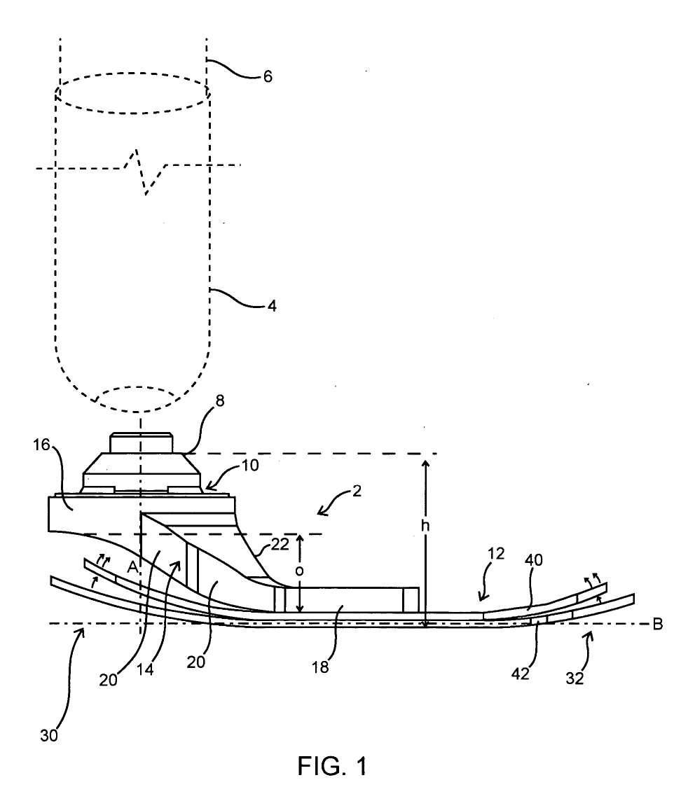

Turning now to FIG. 1, elements of the present invention are shown from the side. Particularly, the figure shows a prosthetic foot 2 and a socket 4 to which it is attached. The socket is adapted to received the remaining limb 6 of a Symes or reverse-Symes amputee.

A distinct or discrete interface member 8 may be provided to join the rest of the foot to the socket. Alternative arrangements or means of interfacing the members may also be provided. Such alternate means of attachment between the foot and socket may be accomplished by way of a number of pins set to interface with corresponding recesses by way of epoxy or adhesive, etc. Naturally, the attachment method, style or precise location may be varied in a manner consistent with remaining aspects of the invention.

Of interest, however, is the low-profile aspect offered by the foot of the invention. It is amendable in applications where the end of the socket (once set in place) to the ground—the distance indicated as the height “h” of the prosthetic—may be a little as about 1 inch (the height available for a patient to stand balanced on an intact foot with an amputation involving an uncut tibia on the other side). Especially outside pediatric applications where larger individuals are concerned, implant height may be about 1½ inches, about 2 inches or possibly more and still accommodate Symes or reverse-Symes amputees.

Even in such a low-profile package, foot 2 preferably includes torsional “ankle” features 10 to allow biased rotation about an axis “A”. Still further, the prosthetic foot may include specialized padding features. For instance, multiple spring rate padding 12, inversion/eversion adaptations and/or a torsion zone may be provided in padding spring(s). Certain variations of the invention may include features of only one sort, another one include a pair of such features, while other variations may include all three options.

A foot base or bracket 14 shaped at least substantially as shown plays a significant role in offering the mutli-faceted functionality elaborated upon below. Bracket 14 comprises a rearward socket mounting zone 16 and a forward padding spring mounting zone 18. An intermediate arch section 20 spans the two other sections. Preferably, the top 22 of the arch offers a transition so as to avoid introduction of stress raisers. The bottom 24 of the arch is configured to allow vertical clearance for spring compression underneath its surface.

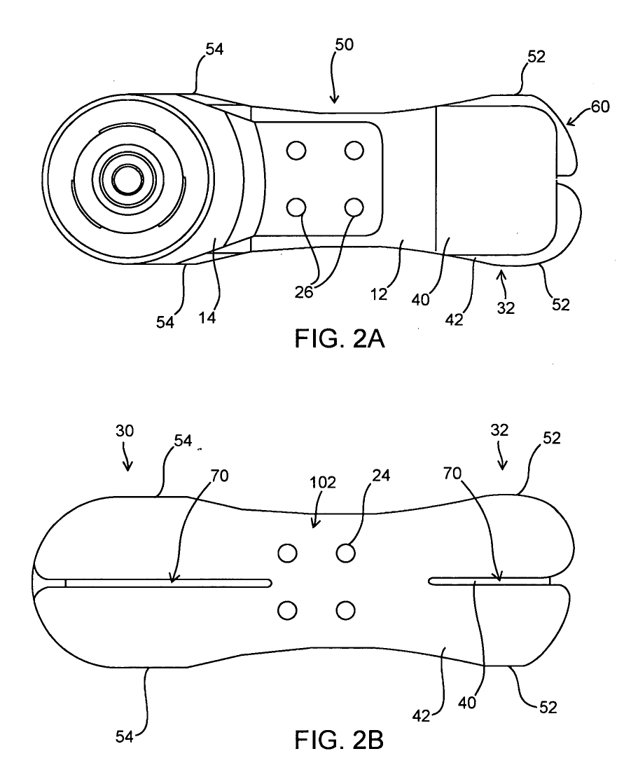

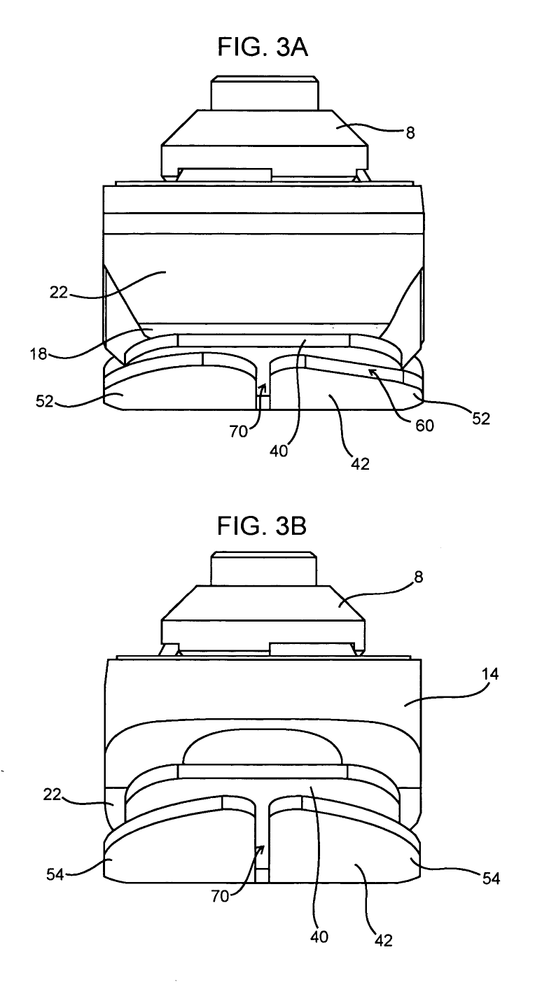

As seen in FIG. 2A, bracket 14 is preferably attached medially with respect to spring padding 12. The attachment may be accomplished using bolts passing through the holes 24 shown in the in underside of the padding and secured by threadings in corresponding holes 26 of the mounting zone in order to sandwich the padding elements between the screw heads or washers and bracket base. Alternatively, nut and bolt sets or other means of attachment may be employed such as press-fit pins, etc. In any case, located medially as shown and a bracket so-configured, the attached padding elements 12 are able to deflect upward both at the heel or rear 30 as well as at the toe or front 32 of the foot.

The head or socket mounting zone 16 bracket preferably incorporates the component parts for torsion zone 10. That is to say, at least certain parts of the assembly are set within the body of the bracket at this point. To provide such a structure, bracket is advantageously machined in aluminum alloy. For lighter weight another alloy may be selected. Still further, alternate methods of manufacture may be employed including casting, composite lay-up or other techniques.

While the various features just touched upon are to be described in further detail below and may be regarded as distinct inventive aspects of the invention, a preferred mode of operation is to employ all the features together. Yet, it is contemplated that the individual features alone form part of the invention, just as do any subsets or combinations that are possible, but not expressly noted.

Multiple Spring Rate Padding

Multiple spring rate padding 12 may be provided by a plurality of leaf springs. While two such springs (40, 42) are pictured, several may be used to finely tune spring rate. As most clearly illustrated in FIG. 1, an upper spring 40 and a lower spring 42 are provided in combination. Each is preferably made of carbon-fiber laminate or another composite material. Alternately, other material having sufficiently high modulus and ability to deflect without failure or plastic deformation may be used (e.g., spring steel or titanium alloy).

The lower or ground-side spring member 42 provides the majority of return force in walking application. Tuned properly, it will slightly miss making contact with the upper spring 40 in normal walking applications. Details of such tuning are described below in connection with the Example of the invention provided.

In running or jumping applications, the lower spring will contact the upper spring. In which case, the springs will move in concert. The degree or amount of compression possible may be a function of the spring rates of the material. Both the arch and rear sections of base 14 will be configured to allow such clearance as desired for full motion of the springs opposite these portions.

Generally, the arch 20 will have a slight concave-down profile as shown in FIG. 1 in order to maximize material use—and yet not interfere with the leaf spring portions which will assume a similar shape upon compression. (Note the arrows indicating such action.) The overall vertical offset “O” between rear section 16 and the base of the front section 18 may vary depending on a number of parameters. Namely, the acceptable height for the desired application, the spring system chosen (e.g., the 2-spring system shown) and possibly other parameters. To enable a dual-spring approach as shown, offset O may typically be between about ½ and about 1 inches in height.

Whatever the spring system chosen, it will be preferred that it is modular in the sense that the spring plates can be changed out (e.g., as a subject grows and requires stiffer springs). In this manner, refitting the foot to a person will not require a new bracket 14 in most instances.

Most typically, the padding members will be located within typical foot apparel for use. To provide clearance for fitting within a shoe, it may be preferred to shorten at least the front or toe region 32 of upper spring 40. To even-out forcing characteristics, the rear or heal section 30 of the same spring may receive similar treatment. Characteristic sizing is shown most clearly in FIGS. 1, 2A and 2B.

Though not shown, it may sometimes be preferred to utilize an asymmetric spring platform (e.g., resulting from a longer toe or heel in the upper spring, variability in material thickness of either spring member and/or varying thickness profile from the toe to the heel.) Often, the spring elements used in the invention will have a constant thickness. Even when symmetry is desired fore and aft of bracket attachment portion 18, leaf spring thickness may be varied. Further, variation in curvature in either or both members 40 and 42 may be applied to the spring design. For example, it may be desired to shape the springs in a concave-down fashion in order to allow for bolt clearance opposite section 18. While such an exemplary variation for the springs is not shown, the ends of each of the springs are shown in a concave-up configuration. One reason for this tracks the reason why shoes are concaved upwards. Namely, when we walk, the swing of the foot is roughly a pendulum like swing about the knee joint. Thus we can have a uniform radius and the foot can swing freely without the toe hitting the floor while you swing your foot in front with a concave-up surface leading this motion. Further, in a dual/multiple spring application as shown, the concave-up arrangement allows for a more uniform/simultaneous contact between spring members 40 and 42 when the prosthetic foot is used for running. As for the top-down shape of the springs, at least the ground-side spring preferably includes an hour-glass shape. The utility of this configuration is described in detail below.

However configured, the spring members serve dual purposes. For one, they allow controlled plantarflexion and dorsiflexion in the foot. In a sense, the forward cantilevered spring member(s) provide return force as would by transmitted through an intact foot connected to an Achilles tendon. Many prosthetic foot devices are designed to provide biased movement only in response to dorsiflexion. By virtue of both forward and rear-facing cantilevered spring member(s), the prosthetic foot of the present invention offers biasing for each degree of freedom, thereby more closely resembling an organic foot in performance.

The second purpose the spring(s) serve is in returning stored energy. Efficient energy return is critical to achieving a normal gait. In order to return energy in a useful fashion, it has been determined that the release of stored energy must be progressive. In other words, impulse-like loading and unloading of a prosthetic foot does little good in achieving satisfactory results.

Too stiff a spring within a prosthetic foot is therefore something to be avoided. Yet (especially to handle higher-force applications ranging from descending stairs to running or jumping), a prosthetic foot must handle forces of upwards of two to three times the weight of a subject. The subject invention preferably handles this need through the multiple-stage spring approach shown.

In contrast, systems using cantilevered leaf spring members for foot padding typically use only a single-stage spring arrangement. Separate spring elements are provided in some products (such as the Modular III™ product from Flex-Foot) to handle plantarflexion and dorsiflexion, but at no time do they provide a cumulative effect to handle high-force situations. In fact, with the Modular III™ product, diverse applications such as running and walking are handled by changing-out the prosthetic foot. Like the Flex-Foot product, the TruStep™ and TruPer™ prosthetic feet offered by College Park Industries uses a pair of discrete spring elements, one to handle plantarflexion and one to handle dorsiflexion. These, however, take the form of discrete rubber/urethane bumpers that may be changed-out to customize performance to tune gait. Some flexure in toe and heel members sprung by the spring elements will occur, but such flexure neither handles the forces associated with walking alone, nor does the flexure provide for additional significant compression of the foot to handle higher stresses and energy return for running applications.

Various prosthetic foot products from Springlite include a plurality of spring elements. Variations using upper and lower canteleavered sections are sold including the Advantage LP,™ Lo Rider™ and Luxon Max™ models. These, however, include a urethane webb between each component, tying their action together at all times. In contrast (as stated variously throughout), the present system described preferably uses a first spring element supplemented by at least one other element, but only when sufficient force is applied. Progressive spring force is added only after a limit is reached and contact between elements occurs.

As noted above, each spring element in the present invention is advantageously constructed from high strength carbon fiber composite (e.g., T300 material). Regarding further detail, in order to maximize plantar flexion and dorsiflexion components relative to other component movements (since such action is the primary sort desired for the spring members), most fibers in the composite should be oriented at 45° with respect to an axis “B” between the pads' front and rear portions. If any bolt holes are provided for attaching the springs to the foot base, additional plies of fibers running at various angles should be included to handle shear stresses. Naturally, as noted above, other attachment approaches may, however, be utilized (e.g., ribs interfacing with complimentary slots). Inversion and/or Eversion Adaptations

Two distinct, but complimentary, features controllably provide for foot inversion and eversion. The first has to do with the planform or top-down shape of the ground-side spring. Namely, it preferably has a waist section 50 inset from adjoining heel 30 and toe 32 sections (i.e., it has a sort of hour-glass shape). Such a configuration results in toe and heel lateral extensions (52, 54) serving as lever arms for imparting and recovering torsional energy in spring 42. They are able to act in such a manner in that the extensions 52, 54 are set latterly of the slimmer mounting section 102 at the effective corner regions of the padding. Where the padding is not specifically adapted to fit within common footwear by providing curved end features as shown, the extensions may terminate in true “corners” or points to increase the available lever arm of each member. In any case, the shape and location of the extensions may vary.

While it is preferred, the ground side spring (possibly the only spring in instances where the above-described features are not utilized) need not be wholly balanced with respect to torsional extensions. It may be desired to include only one, two or three extensions instead of all four. Still further, even where extensions are provided, they may be asymmetrical. Note, for instance, the asymmetry between the side of toe section 32 of the foot. Angled profile 60 is provided, again, to facilitate the use of common footwear. Accordingly, the prosthetic foot shown is meant to function as a left-side foot.

Regardless of such details, a feature that works well in combination with the extension(s) is an optional split region 70 in either or both of the toe and heel sections. The split increases compliance to torsional loading by the extensions in a manner that will be apparent to one with skill in the art.

Note, however, that even without extension sections (or a corresponding waist section 50), splitting at least the ground side spring platform into one or more regions offset regions allows for better response to off-center loads. Such ability increases the compliance of the foot relative to uneven topology. Accordingly, providing a split toe and/or split heel section allows in some way for padding inversion and eversion in relation to the ground.

Biased Torsion Zone

Provision for torsional freedom in a prosthetic foot is not uncommon. Most typically, however, nothing more than bearings are provided to enable unbiased rotation to simulate ankle functionality. The present invention offers biased rotational features instead. Certain activities such as hitting in baseball or tennis involve wind-up and release type action at the athlete's ankle. For an amputee to be most successful in accomplishing such activity energy storage and return offers a great advantage.

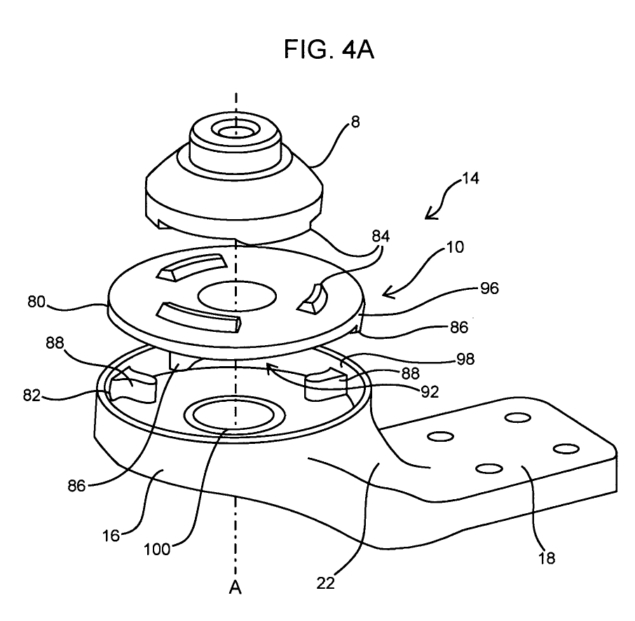

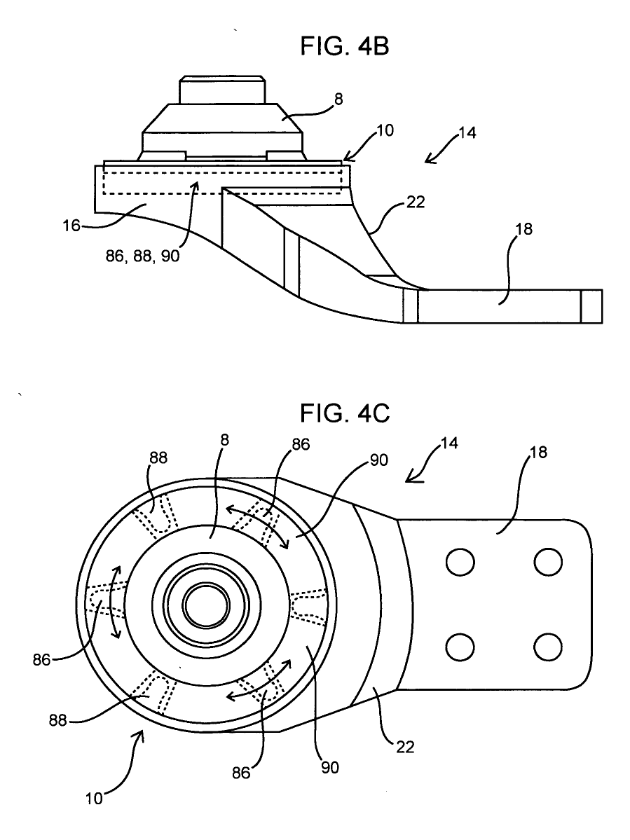

FIGS. 4A-4C illustrate a preferred manner of providing such functionality. A rotor 80 and stator 82 combination is provided. As shown, the stator is machined into bracket member 14 in the form of a recess. Upon assembly as indicated in FIG. 4A, interface member 8 and rotor 80 are fixed in position relative to each other by stop members 84 that interfit as shown in FIG. 1. FIG. 4A also clearly shows vanes 86 and 88 in the rotor and stator, respectively. As indicated in FIG. 4C, electrometric bumper or wedge members 90 are set between the vanes. The vanes compress certain of the wedges depending on the direction of rotor rotation. When cast in place or otherwise adhered to the vanes, the bumpers that are not compressed may be stretched and held in tension upon such rotation. Whatever the case, at least some of the bumpers will provide return force to a neutral position. Furthermore, it should be understood that while the stator and rotor vanes are shown in FIG. 4C to be set to use equal size wedge members 90, they may be offset so as to employ different sized bumpers. This may be advantageous in order to limit motion in one rotational direction and/or offer differential biasing. Still further, an approach using different hardness/durometer bumpers on either side of one or more of rotor vanes 86 may be used to similar effect.

Various bearing arrangements may be employed in torsion zone 10. For example, rotor 80 may rest on a rubber disk which houses several roller bearings. Alternately a base 92 of the rotor between the vanes my simply be set against a busing set within recess 94 of the foot bracket. A through-bolt running along axis A easily secures the various components shown together. Of course, as various design possibilities may be applied to in the case of bearing design, so may there be regarding assembling and securing the inventive prosthetic.

Still further, in addition to the approach pictured, other approaches for torsion zone 10 are envisioned. For instance, the spring type may be changed and the vanes eliminated to accommodate such modification. Most advantageously, however, the torsion zone will have a low profile, such as on the order of less than ½ inch in height, to facilitate use of the prosthetic foot described herein by Symes-type amputees.

This low-profile design is evident in FIG. 4B. Here, the flange of rotor 80 is at least partially received within the stator section 82. The vanes and bumper members are set below that level, and thereby encapsulated. In order to seal the section, it may be desired to set an O-ring or another sealing means around the girdle 96 of the rotor or stator wall 98 above the vanes. However, no such provision is required and is therefore optional as are all those features discussed above in terms of optional, preferential and/or permissive language.

### EXAMPLE

For a 65-75 lb, 12-year old male, a custom prosthetic foot was developed. In this device each of the features providing biased movement for prosthetic foot torsion, inversion-eversion, plantarflexion, dorsiflexion, heel strike and toe-off were used. The device did not include the torsion features described. As such, the testing only covered creation of the optional features of the invention.

Yet, in customizing the prosthetic foot to the extent possible in view of the hardware available certain test data regarding the subjects gait was either derived or measured directly. Namely, using force plates in a biomotion lab, the values for the forces and moments generated at the ankle were recorded along with the ankle angle, foot angle for different activities such as walking, stair climbing & running. See, “A point cluster method for in vivo motion analysis: applied to a study of knee kinematics,” J Biomech Eng. 1998 December;120(6):743-9.

For a 30° amount of plantarflexion (an amount similar to the position encountered for toe strike while running) with one prototype of leaf springs, engagement of the ground-side spring with the upper spring occurred at about 500N force and 1.7 cm of vertical displacement. Considering this force corresponding to somewhat less than the subject's body weight, it was determined that the bottom leaf spring of a second prototype should be close to twice as thick. The thickness was so-increased despite the spring load bearing capability actually increasing as the square of thickness, to account for the increasing weight and strength of the pediatric subject. This design decision offered a longer-term fit to the user. Of course, a more conservative adjustment could also have been made instead.

Even though the testing proved that padding spring adjustment was called for, the upper spring supplemented the force of the lower spring after contact was made between as desired. In the case tested, the two members made contact at roughly 500N of force application. As contact progressed through compression and the spring members moved relative to each other, the leading edge portion of the upper member receded relative to the lower spring member. The resultant increase in mechanical advantage of the lower spring over the upper one produced a gradual decrease in spring rate after reaching a substantially linear zone in the range of between about 800N and 1800N of compressive force. Such interaction of elements helped to avoid force spiking or a steeply geometric spring rate, thereby enabling excellent work force/energy return.

The test data also demonstrated the durability of the design. Even at 3000 N of force applied by a test machine, no sign of failure was observed in any of the system components. Full patency was similarly observed in connection with a setup applying equal force at 45° of plantarflexion.

Feedback by the recipient of the prosthetic foot was positive as would be expected in view of the manner in which it was specifically tuned to meet his particular requirements. On a basic level, such qualitative feedback and confidence is important.

In addition to such feedback, however, quantitative measurements were taken to gauge the performance of the prosthetic foot. Gait data was obtained through motion analysis under a variety of conditions. These conditions included normal walking, fast walking, jogging, ascending stars and descending stairs. Performance of the subject's unaffected leg was compared to the performance by the amputated leg—first in connection with the Springlite prosthesis the subject was accustomed to, and then with a prototype including each of the features described above (again, minus the torsion zone). The results were positive after only a short period of time.

Subsequent testing in a prosthetic foot including the torsion features of the invention was later attempted. The noted subject expressed interest in the torsion features as potentially enabling him to better participate in sports and engaging on other physiological movements. However, failure of the socket/foot interface of the design used (a design different from the improved implementation shown in the figures) resulted in the termination of the testing prior to acquiring any quantitative data at the lab in which the attempted testing occurred. The present design offers a dramatically improved foot/socket interfaces designed to handle the rigors of the biased torsion features offered by the invention.

### Claims

Though the invention has been described in reference to a single example, optionally incorporating various features, the invention is not to be limited to the set-up described. The invention is not limited to the uses noted or by way of the exemplary description provided herein. It is to be understood that the breadth of the present invention is to be limited only by the literal or equitable scope of the following claims.

1. A prosthetic foot system, said system comprising:
a foot, wherein said foot comprises a base having a rear section for attachment to a socket, a forward section for attachment to at least one padding spring and an arch section spanning said front and rear sections,
wherein said at least one padding spring extends forward and rearward of said forward base section,
wherein said arch and said base rear section are configured to provide clearance for upward deflection of said at least one padding spring opposite thereto, and
wherein said rear section comprises a recess serving as a stator having a plurality of vanes, a rotor having a plurality of vanes received within said recess, and a plurality of spring elements, each said spring element between opposing rotor and stator vanes thereby providing torsional features for said foot.

2. The system of claim 1, wherein said foot further comprises an interface member for attachment between said rotor and said socket.

3. The system of claim 2, further comprising said socket attached to said interface member.

4. The system of claim 1, wherein sad at least one padding spring consists of an upper and lower leaf springs.

5. The system of claim 4, wherein said springs are configured so only said lower spring is used in walking.

6. The system of claim 4, wherein said springs are configured so said upper spring provides supplemental force to said lower spring in running or jumping.

7. The system of claim 4, wherein said upper spring has ends inset from ends of said lower spring.

8. The system of claim 1, wherein said at least one padding spring has at least one of a split toe or a split heel.

9. The system of claim 1, wherein said at least one padding spring has at least one lateral extension.

10. The system of claim 1, wherein said foot, and an interface member for connecting said foot to a socket has a height of between about 1 and about 2 inches.

11. A prosthetic foot comprising:
a base having a rear section for attachment to a socket, a forward section for attachment to at least one padding spring and an arch section spanning said front and rear sections,
said at least one padding spring extending forward and rearward of said forward base section,
said arch and said base rear section being configured to provide clearance for upward deflection of said at least one padding spring opposite thereto, and
said rear section comprising a stator having a plurality of vanes, a rotor received at least partially within said stator and having a plurality of vanes, and a plurality of spring elements, each said spring element between opposing rotor and stator vanes thereby providing torsional features for said foot.

12. The prosthetic foot of claim 11, wherein said stator is formed in a single piece of base material, together with said arch and forward sections of said base.

13. The prosthetic foot of claim 11, further comprising an interface member adapted to be affixed to said rotor and for attachment to a socket.

14. The prosthetic foot of claim 11, having a height of between about 1 and about 2 inches.

15. The prosthetic foot of claim 11, wherein sad at least one padding spring consists of an upper and lower leaf springs.

16. The system of claim 15, wherein said springs are configured so only said lower spring is used in walking.

17. The system of claim 15, wherein said springs are configured so said upper spring provides supplemental force to said lower spring in running or jumping.

18. The system of claim 15, wherein said upper spring has ends inset from ends of said lower spring.

19. The system of claim 11, wherein said at least one padding spring has at least one of a split toe or a split heel.

20. The system of claim 11, wherein said at least one padding spring has at least one lateral extension.
